(19) United States
(12) Patent Application Publication
(10) Pub. No.: US 2016/0113987 A1
(43) Pub. Date: Apr. 28, 2016

(54) NUTRITIONAL SUPPLEMENTS

(71) Applicant: VitaThinQ, Inc., Davie, FL (US)

(72) Inventors: Donald M. Choi, Davie, FL (US); Edwin Peissis, Davie, FL (US)

(21) Appl. No.: 14/924,095

(22) Filed: Oct. 27, 2015

Related U.S. Application Data
(60) Provisional application No. 62/068,881, filed on Oct. 27, 2014.

(51) Int. Cl. A61K 36/88; A61K 36/38; A61K 36/74; A23G 3/42; A61K 36/68; A23G 3/36; A23G 3/54; A23G 3/48; A61K 31/522; A61K 31/4745

(52) U.S. Cl. CPC A61K 36/88; A61K 31/522; A61K 36/38; A61K 36/74; A61K 31/4745; A61K 36/68; A23G 3/36; A23G 3/54; A23G 3/48; A23G 3/42; A23G 3/364; A23V 2002/00

(57) ABSTRACT

Compositions are provided for preparing low dosage supplements in the form of a confectionery which are easy to swallow and have a desirable taste. The resulting compositions are suitable for delivering all natural active ingredients to aid in alleviating a particular disease or promote overall well-being. Methods of forming the confectionery are also provided herein.

### CROSS REFERENCE TO RELATED APPLICATIONS

The present application claims priority to and the benefit of U.S. Provisional Application No. 62/068,881 filed on Oct. 27, 2014, the entire contents of which is hereby incorporated by reference.

### TECHNICAL FIELD

The present disclosure relates to confectionery compositions useful in delivering nutrients to the body, for alleviating or targeting certain ailments and/or promoting a healthy well-being. More particularly, the present disclosure relates to compositions which deliver nutritional supplements via confectionery compositions that include all natural ingredients derived from plants or animals.

### BACKGROUND

Consumers have become increasingly conscious of the ingredients contained in nutritional supplements possessing vitamins, minerals, herbs, and other dietary ingredients. These supplements may be used to assist a person in meeting certain dietary needs, alleviate ailments, and/or promote overall well-being. However, many dietary supplements are made with chemicals or man-made ingredients which may have harmful effects on the body. These supplements may have a flavor and/or mouthfeel, i.e., a product's physical and chemical interaction in the mouth, which make them unpalatable and leave an unpleasant aftertaste in the mouth.

In addition, current commercially available nutritional supplements do not offer the plethora of controlled release delivery systems that are available for drugs. Studies have demonstrated that some of the most commonly used multivitamins and drugs on the market are filled with binders and fillers that make them difficult to digest, and therefore reduce their effectiveness because the body is only absorbing about 40-60% of an active ingredient. This is despite the fact that it is well known to those skilled in the art that controlled release formulations, which are effective in maintaining nutraceutical blood levels over extended periods of time, result in optimal supplementation.

Nutritional supplements are known as nutraceuticals when they have a proven benefit to the structure or function of a body organ or system. As the benefits of nutraceuticals are often observed at high doses, large quantities of a controlled release formulation may make their use unacceptable.

Numerous methods of administering these supplements have been developed, such as tablets, hard and soft shell capsules, effervescent wafers, liquids, gummy chews, sublingual pellets, and the like. However, many individuals find it difficult to chew gummy chews or difficult to swallow tablets and/or capsules, while others object to the high refined sugar content or low nutritional content of such dosage forms. Furthermore, large quantities of a liquid supplement may be inconvenient for transport, dosing and/or consumption.

Improved methods of providing palatable, all natural dietary supplements, which possess good mouthfeel and do not leave an unpleasant aftertaste, remain desirable. A controlled release formulation that contains only small amounts of formulation components, other than the target nutraceutical, would also be desirable, not only because it would reduce the frequency of dosing for enhanced user convenience and compliance, but it would also reduce the severity and frequency of gastric difficulties as it maintains substantially constant blood levels and avoids the need for large and repeated doses of immediate release formulations required to achieve the benefits reported in the literature. Additionally, alternative methods of administration that do not require swallowing a tablet, pill, capsule, and the like, remain desirable.

### SUMMARY

The present disclosure provides an all-natural delivery system including a quick dissolving, chewable, confectionery supplement, and an alternative method to deliver natural supplements for the body to absorb in low dosages with a pleasant taste and good mouthfeel. The supplement's low dosage allows a user to administer the right amount of dosage for their needs with little to no side effects or drug toxicity while maximizing a treatment's impact. Specifically, the compositions of the present disclosure can be utilized to aid in alleviating various ailments such as, but not limited to, obesity, high cholesterol, depression, dry mouth, osteoporosis, colds, memory loss/Alzheimer's disease, dysfunctional liver, erectile dysfunction, fatigue and/or detoxification. The compositions of the present disclosure may also promote overall well-being.

The present disclosure provides a composition including at least one natural sweetener; at least one binding agent; at least one thickening agent; at least one stabilizing agent; at least one flavoring agent; at least one natural active ingredient; and optionally at least one solvent.

The present disclosure also provides a composition for forming a confectionery supplement including at least one natural sweetener in amounts from about 60% to about 95% by weight of the composition; at least one binding agent in amounts from about 0.1% to about 3% by weight of the composition; at least one thickening agent in amounts from about 1% to about 8% by weight of the composition; at least one stabilizing agent in amounts from about 0.1% to about 5% by weight of the composition; at least one flavoring agent in amounts from about 0.1% to about 10% by weight of the composition; at least one natural active ingredient in amounts from about 0.5% to about 25% by weight of the composition; and at least one solvent in amounts from about 1% to about 20% by weight of the composition.

Methods of producing dietary supplements are also provided herein. In embodiments, a method of the present disclosure includes combining at least one binding agent in at least one solvent to form a base solution; combining at least one natural sweetener, at least one thickening agent, at least one stabilizing agent, at least one flavoring agent, and at least one natural active ingredient to form a dry mixture; combining the dry mixture with the base solution to form a doughy paste; rolling the doughy paste to a desired thickness; cutting the rolled doughy paste into a desired shape; drying the shaped doughy paste until hardened to a desired hardness to form a hardened dough; applying at least one sub coating layer to the hardened dough to form a confectionery supplement; and optionally applying at least one outer layer to the sub coating layer to form a coated confectionery supplement.

### DETAILED DESCRIPTION

The present disclosure provides a composition or delivery system capable of administering a nutraceutical at a low dose and methods of nutritional supplementation that enables better dosage control and distribution together with a pleasant flavor and mouthfeel, without producing an unpleasant aftertaste. The presently disclosed delivery systems control the rate at which a drug or active is released and the location in the body where it is released.

The presently disclosed nutritional supplement is an oral confectionery unit that dissolves in the mouth and/or on the tongue. The confectionery nutritional supplement of the present disclosure may be in the form of a variety of shapes including a pellet, a tablet, a capsule, a ball, a pillow, a chunk, a stick or a slab, among others. In some embodiments, the confectionery nutritional supplement may be in powder form, granules, syrups, gels, hard and semi-hard chews, gummies, lingual strip, sublingual strip, oral mist, rapidly disintegrating tablet, lyophilized wafer, chewing gums, chocolate or a drink. In embodiments, pellet forms may be obtained where a process is used to make a doughy paste, which is then baked and dried to harden via a rolling and/or panning process. In embodiments, the composition and method of administration do not require swallowing of a solid or liquid dosage form and may dissolve when placed in the mouth upon contact with saliva in the oral cavity, i.e., on the tongue. In embodiments, the oral formulations provide rapid absorption after introduction to the oral cavity, followed by buccal and/or sublingual absorption. In embodiments, the pellet forms of the compositions of the present disclosure provide ease of storage, transportation, and usage in contrast with a liquid form of a supplement.

In embodiments, the compositions of the present disclosure may aid in alleviating certain ailments and diseases, including but not limited to, obesity, high cholesterol, depression, dry mouth, osteoporosis, colds, memory loss/Alzheimer's, liver or kidney disease, erectile dysfunction, calcium and vitamin deficiency, fatigue and/or maintaining blood glucose sugar levels, pain management, symptoms caused by cancer and aids, among others. In other embodiments, the compositions of the present disclosure may be utilized to promote the overall well-being of the user such as, but not limited to, detoxification, enhancing digestion, enhancing immunity, enhancing sleep, and quitting smoking, among others.

In embodiments, all of the components utilized in the confectionery supplements of the present disclosure are naturally derived ingredients extracted from plants, e.g., from raw plant parts such as leaves, roots, seeds, and animal extracts. The ingredients are obtained from organically grown plants and are free of any genetic modification (GMO free). In embodiments, the compositions do not contain any chemicals or preservatives.

In embodiments, the compositions of the present disclosure comprise a core including the nutritional supplement(s) and a coating that controls release of the supplement from the dosage form. In embodiments, the main component of the core of the composition includes a sweetener or sugar and works to preserve the composition to provide a long shelf life. Any natural sweetener within the purview of those skilled in the art may be used in accordance with the present disclosure. In embodiments, the natural sweetener may include organic evaporated cane sugar, beet sugar, stevia, sucrose, dextrose, fructose, glucose, corn syrup, maltose, maltodextrin, lactose, maple syrup, molasses, honey, barley malt, rice syrup, honey, agave nectar, fruit juice sweeteners, and the like, and combinations thereof, all of which are readily available in bulk from commercial manufacturers. Other natural sweeteners, such as xylitol, Truvia®, or Splenda® may be used as long as they are considered natural by the Federal Drug Administration (FDA).

The sweetener may be in the form of a granulate or powder. In embodiments, the sweetener may include organic evaporated cane sugar. The cane sugar may be a granulated grade of cane sugar, with from about 90% to about 95% of the particles having a particle size from about 10 to about 450 microns, in embodiments from about 250 to about 400 microns, with an approximate mean particle size of about 320 microns. In other embodiments, the evaporated cane sugar may be a powdered grade, with from about 80% to about 90%, in embodiments about 85%, of the particles having a particle size from about 10 to about 500 microns, in embodiments from about 100 microns to about 400 microns, with a mean particle size of about 150 microns.

In embodiments, the sweetener includes organic evaporated cane sugar in combination with an all-natural high intensity sweeter such as, for example, stevia. Stevia is one of several non-caloric high-intensity natural sweeteners from the plant Stevia rebaudiana bertoni which produces a number of sweet compounds collectively referred to as steviol glycosides, making stevia 300 times sweeter than sucrose alone. These steviol glycosides can be extracted from the plant using a variety of known natural product extraction processes. These naturally-occurring sweeteners are heat stable, pH stable, do not ferment, and do not induce a glycemic response in humans. Although stevia and stevia plant extracts have a strong bitter component making their use in sweeteners more challenging, the compositions of the present disclosure are specially formulated to mask any such bitterness taste. In embodiments, one particular extract utilized in the present composition, rebaudioside A (Reb A), is a non-caloric sweetener with functional and sensory properties much superior to those of many other non-caloric sweeteners, and which in processed form can be 70 to 400 times more potent than sugar.

In embodiments, the sweetener may be present in the composition in an amount from about 60% to about 95% by weight of the composition, in embodiments from about 65% to about 90% by weight of the composition. In embodiments, where utilized, the high intensity sweetener may be present in the composition in an amount of from about 0.001% to about 3% by weight of the composition, in embodiments from about 0.01% to about 1% by weight of the composition.

Any suitable binding agent may be used in the composition of the present disclosure to help supplements bind with the sweetener. In embodiments, a binding agent may include gelatin, carrageen, seaweed, locust bean gum, pectin, alginate, agar, guar gum, xanthan gum, gum arabic, starch derivatives, isomalt, and the like, and combinations thereof.

In embodiments, the binding agent includes gelatin having a gel strength or bloom value of about 150 to about 175 to assist with increasing the thermal stability of the composition. In embodiments, the binding agent may be present in an amount from about 0.1% to about 3% by weight of the composition, in embodiments from about 0.3 to about 2% by weight of the composition.

In embodiments, the binding agent may be combined with a solvent to dissolve the binding agent to form a base solution and provide fast delivery of the active ingredients. Acceptable solvents of the present disclosure include water and/or sugar syrup, including but not limited to, white rice syrup, rice flour, rice starch, sucrose syrup, brown rice syrup, tapioca syrup, corn syrup, agave syrup, stevia syrup, and the like, and combinations thereof. The solvent may be present in an amount from about 1% to about 20% by weight of the composition, in embodiments from about 2% to about 10% by weight of the composition.

The compositions of the present disclosure may include a stabilizing agent. In embodiments, a stabilizing agent may include gelatin, carrageenan, seaweed, locust bean gum, pectin, alginate, agar, guar gum, xanthan gum, gum arabic, EmulTru™ starch, maltodextrin, shellac, shellac gum, ascorbic acid, benzoic acid, fumaric acid, starch derivatives, and the like, and combinations thereof. In embodiments, powdered gum arabic may be utilized as the stabilizing agent. In embodiments, the stabilizing agent may be present in an amount from about 0.1% to about 5% by weight of the composition, in embodiments from about 1% to about 3% by weight of the composition.

The compositions of the present disclosure may also include a thickening agent. Suitable thickening agents utilized to thicken the consistency of the composition may include white rice syrup, rice flour, rice starch, sucrose syrup, brown rice syrup, tapioca syrup, corn syrup, agave syrup, stevia syrup, and the like, and combinations thereof. In embodiments, the thickening agent may be present in an amount from about 1% to about 8% by weight of the composition, in embodiments from about 2% to about 6% by weight of the composition.

In embodiments, white rice syrup may be utilized as the thickening agent. Rice syrup contains only half the sweetness of common refined sugar. Rice syrup is a sweetener made from whole grain rice that has been cultured and fermented with enzymes to break down the natural starches into complex carbohydrates and more simple carbohydrates, including maltose and glucose. This mixture of simple and complex carbohydrates enables the sugars to be absorbed more slowly by the digestive system, resulting in a more steady level of blood glucose levels than obtained with other sweeteners. The glucose is absorbed immediately, while the maltose and complex carbohydrates absorb over several hours.

In embodiments, the rice syrup is lightly-filtered, non-clarified white rice syrup with a dextrose equivalent level (D.E.) of 26 (as compared with corn syrup which is 100 D.E.), although rice syrup with different D.E. levels may be substituted (the higher the D.E. level, the sweeter the syrup). In embodiments, the sugar profile of the rice syrup is: glucose-6 g/100 g; maltose-13 g/100 g; other carbohydrates-57 g/100 g.

In embodiments, flavoring agents may be added to the compositions of the present disclosure to mask any unpleasant tastes of certain supplements and active ingredients included in the composition. Flavorants may include those flavors within the purview of those skilled in the art, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, cassia oil, and the like, and combinations thereof. Also useful flavorings include artificial, natural and synthetic fruit flavors such as citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya, watermelon, and the like, combinations thereof.

Other potential flavors which can be used include milk flavor, butter flavor, cheese flavor, cream flavor, and/or yogurt flavor; vanilla flavor; tea or coffee flavors, such as green tea flavor, oolong tea flavor, and the like; cocoa flavor, chocolate flavor; mint flavors, such as peppermint flavor, spearmint flavor, and Japanese mint flavor, and the like; spicy flavors, such as asafetida flavor, ajowan flavor, anise flavor, angelica flavor, fennel flavor, allspice flavor, cinnamon flavor, camomile flavor, mustard flavor, cardamom flavor, caraway flavor, cumin flavor, clove flavor, pepper flavor, coriander flavor, sassafras flavor, savory flavor, Zanthoxyli Fructus flavor, perilla flavor, juniper berry flavor, ginger flavor, star anise flavor, horseradish flavor, thyme flavor, tarragon flavor, dill flavor, capsicum flavor, nutmeg flavor, basil flavor, marjoram flavor, rosemary flavor, bayleaf flavor, and/or wasabi (Japanese horseradish) flavor, and the like; floral flavors; sour flavors, such as flavors formed from malic acid, citric acid, and/or ascorbic acid, and the like; and/or vegetable flavors. Commonly used flavors include mints such as peppermint, menthol, spearmint, berry mint, ginger mint, ginseng mint, green tea mint, artificial vanilla, cinnamon derivatives, and various fruit flavors, whether employed individually or in admixture. Flavors may also provide breath freshening properties, particularly the mint flavors.

In embodiments, the compositions of the present disclosure may include a peppermint flavor. In embodiments, the peppermint flavor may be triple distilled. In other embodiments, the compositions of the present disclosure may include cocoa extract (available commercially from a number of vendors e.g., PROCAO™ cocoa extract commercially available from PROVA® located in France) either alone or in combination with peppermint flavoring.

The flavoring agent may be present in amounts from about 0.1% to about 10% by weight of the composition, in embodiments from about 1% to about 5% by weight of the composition.

In some embodiments, the flavoring agent may be employed in liquid and/or dried form. Suitable drying means such as spray drying the liquid may be used. Alternatively, the flavoring agent may be powdered, beaded, absorbed onto water soluble materials, such as cellulose, starch, sugar, maltodextrin, gum arabic and so forth, or the flavoring agent may be encapsulated.

Typically, encapsulation of a component will result in a delay in the release of the predominant amount of the component during consumption of the confectionery composition that includes the encapsulated component (e.g., as part of a delivery system added as an ingredient to the confectionery composition). In some embodiments, the release profile of the ingredient (e.g., the flavor, sweetener, active, etc.) can be managed by managing various characteristics of the ingredient, the delivery system containing the ingredient, and/or the confectionery composition containing the delivery system and/or how the delivery system is made.

In embodiments, the composition of the present disclosure may include an active ingredient (sometimes referred to herein as “actives”). Actives generally refer to those ingredients that are included in a delivery system and/or confectionery composition for the desired end benefit they provide to the user. In some embodiments, actives can include nutrients, nutraceuticals, vitamins, herbals, nutritional supplements, and combinations thereof.

Suitable examples of natural active ingredients utilized in the compositions of the present disclosure may include anti-cholesterolemics, analgesics, anti-convulsants, anti-depressants, anti-diabetic agents, anti-diarrhea preparations, anti-histamines, anti-hypertensive drugs, anti-inflammatory agents, anti-lipid agents, anti-manics, anti-nauseants, anti-stroke agents, anti-viral agents, anti-acne agents, alkaloids, amino acid preparations, anti-tussives, anti-infective agents, appetite suppressants, appetite stimulants, blood modifiers, bone metabolism regulators, cardiovascular agents, central nervous system stimulants, decongestants, dietary supplements, enzymes, fertility agents, gastrointestinal agents, homeopathic remedies, migraine preparations, memory boosting agents, motion sickness treatments, muscle relaxants, osteoporosis preparations, psychotherapeutic agents, respiratory agents, sedatives, urinary tract agents, vasodilators, laxatives, antacids, expectorants, anti-anxiety agents, anti-ulcer agents, anti-inflammatory substances, stimulants, anti-nauseants, diuretics, and combinations thereof.

In embodiments, the active ingredients may be present in the composition in amounts from about 0.5% to about 25% by weight of the composition, in embodiments from about 1% to about 20% by weight of the composition. In embodiments, the active ingredient may be present in the composition in amounts from about 100 mg to about 120 mg.

In embodiments, the confectionery supplements of the present disclosure may include active ingredients which aid in increased alertness and provide increased energy. In embodiments, the active ingredients may include caffeine extract, guarana, ginseng, ginko, yerba mate, green tea extract, taurine, kola nut extract, rhodiola root extract, minerals such as, for example, iodine, among others, vitamins such as for example vitamin B12, niacin, and vitamin D, among others, amino acids, and combinations thereof. In embodiments, the active ingredient may be caffeine extract. The caffeine extract may be in encapsulated form. In embodiments, the active ingredient, such as encapsulated caffeine, may be present in amounts from about 0.5% to about 10% by weight of the composition, in embodiments from about 1% to about 8% by weight of the composition.

In embodiments, the confectionery nutritional supplements of the present disclosure may include active ingredients which aid in weight loss or suppress appetite. The active ingredients may include garcinia cambogia, coffea robusta, green coffee extract, saffron extract, raspberry ketones, caffeine, green tea extract, hoodia gordonii, milk thistle, green tea, calcium, konjac root, reishi mushroom, fish Oil, gymnema sylvestre, guggul, brindleberries, chitosan, whey protein, beta glucan, kunjo, African mango, alpha-cyclodextrin, iodine, vitamin D, n-acetyy-tyrosine, soy phospholipids, and combinations thereof.

In embodiments, the weight loss composition may include at least one of garcinia cambogia, green coffee extract, and saffron. Garcinia cambogia may be derived from a green fruit pumpkin that contains a natural substance called hydroxycitric acid (HCA), which may inhibit lipase secretion that creates fat cells, and reduces appetite. Green coffee extract contains chlorogenic acid which controls blood sugar, and increases metabolism which will burn existing body fat. Saffron extract may help release serotonin in the brain and reduces emotional hunger.

In embodiments, the active ingredients in the weight loss confectionery composition are present in amounts from about 5% to about 25% by weight of the composition, in embodiments from about 10% to about 20% by weight of the composition. In embodiments, the weight loss confectionery composition provides appetite suppression up to about 50%, in embodiments, from about 25% to about 40%, in embodiments, from about 30% to about 40%.

In embodiments, the confectionery nutritional supplement of the present disclosure may include active ingredients which enhance working memory or boost concentration. The active ingredients may include phosphatidylcholine, caffeine, ginkgo biloba extract, rhodiola root extract, vitamin B6, folic acid, vitamin B12, green tea extract, vinpocetine, theanine, blueberry powder, phosphatidylserine, dimethylaminoethanol (DMAE), pantothenic acid, and combinations thereof. In embodiments, the memory aid may include phosphatidylcholine which is a natural supplement currently used in studies for Alzheimer's disease.

In embodiments, the confectionery nutritional supplements of the present disclosure may include active ingredients which boost immune function. The active ingredients which boost immune function may include vitamin D, vitamin C, zinc, copper, magnesium, garlic, bee pollen, flax seed, nutritional yeast, alfalfa leaf, kelp, milk thistle, turmeric, ginger, thyme, suma, wheat, soy, milk, and combinations thereof.

In embodiments, the confectionery nutritional supplements of the present disclosure may include active ingredients which alleviate dry mouth. In embodiments, the active ingredient for alleviating dry mouth may include jaborandi, an herbal extract. In embodiments, the active ingredients may include jaborandi in combination with the active ingredients utilized in boosting immunity.

In embodiments, the confectionery nutritional supplements of the present disclosure may include active ingredients which repair bone and prevent bone loss. The active ingredients may include calcium, black walnut tincture, vitamin D, and combinations thereof.

In embodiments, the confectionery nutritional supplements of the present disclosure are suitable for administration to those with diabetes. The confectionery supplements may help maintain blood glucose sugar level as well as healthy blood pressure levels.

In embodiments, the confectionery nutritional supplements of the present disclosure may include active ingredients which provide detoxifying benefits for the body. The active ingredients may include green tea extract, acai (Euterpe Oleracea) extract, ginger root, cranberry (Vaccinium Macricarpon) powder, milk thistle (Silybum Marianum) seed extract, fennel (Foeniculum Vulgare) seed, fenugreek (Trigonella Foenum-Graecum) seed extract, peppermint (Mentha×Piperita) leaf, blueberry extract, prunes, flaxseed, chia seeds, fiber, and combinations thereof. In embodiments, the active ingredient includes green tea extract.

In embodiments, the confectionery nutritional supplements of the present disclosure may include active ingredients which may lower cholesterol. The active ingredients may include, Berberine Hydrochloride HCl [97%] (from Indian Barberry Root), Picrorhiza kurroa (from Root Extract in Nepal), and/or other plant extracts within the purview of those skilled in the art. In embodiments, the confectionary supplement comprises Berberine and Picrorhiza kurroa and may reduce LDL cholesterol up to about 30%, in embodiments, from about 20% to about 25%. In embodiments, the proposed mechanism of action of the cholesterol supplements may include phosphodiesterase inhibition, AMP-activated protein kinase (AMPK) activation, regulation of oxidative stress through Quinone Oxidoreductase 2 (QR2) inhibition, or 3-hydroxy-3-methyl-glutaryl-CoA lyase (HMG CoA lyase) reductase modulation.

In embodiments, the confectionery nutritional supplements of the present disclosure may include active ingredients for male vitality, for enhancing muscle growth and/or which alleviate symptoms of erectile dysfunction. The active ingredients may include didehydroepiandrosterone (DHEA), Panax ginseng, guarana extract, horny goat weed, Eurycoma longifolia, Xanthoparmelia Scabrosa, Cnidium Monieri, Mucuna Pruriens, and/or other plant extracts within the purview of those skilled in the art, and combinations thereof.

In embodiments, the confectionery nutritional supplements of the present disclosure may include active ingredients which treat patients with heart disease or rheumatoid arthritis. The active ingredient may include edible gold fragments or flakes and they may be included in the core or coating compositions of the present disclosure in amounts effective to provide a desired result. Gold in small doses may improve intelligence and memory, strengthen the heart muscle, and increase endurance and toning. Gold may also decrease risk of heart attacks, epilepsy, and diseases of the lungs and liver. In embodiments, the gold may be powdered or granulated. In embodiments, the gold may be edible 23-carat gold. In embodiments, the gold may be encapsulated to provide a desired rate of release.

Embodiments of the present composition may include at least one natural sweetener in an amount from about 60% to about 95% by weight of the composition; at least one binding agent in an amount from about 0.1% to about 3% by weight of the composition; at least one thickening agent in an amount from about 1% to about 8% by weight of the composition; at least one stabilizing agent in an amount from about 0.1% to about 5% by weight of the composition; at least one flavoring agent in an amount from about 0.1% to about 10% by weight of the composition; at least one natural active ingredient in an amount from about 0.5% to about 25% by weight of the composition; and at least one solvent in an amount from about 1% to about 20% by weight of the composition.

The compositions of the present disclosure may also include vitamins, minerals, herbs, and other dietary ingredients, and combinations thereof. Vitamins that may be used in accordance with the present disclosure include, for example: vitamin A from sources such as vitamin A palmitate, vitamin A acetate, beta carotene, combinations thereof, and the like; vitamin C from sources such as sodium ascorbate, ascorbic acid, calcium ascorbate, combinations thereof, and the like; vitamin D from sources such as ergocalciferol (D2), cholecalciferol (D3), combinations thereof, and the like; vitamin E from sources such as d-α-tocopheryl acetate, d-α-tocopherol, d-α-tocopheryl succinate, dl-α-tocopherol, dl-α-tocopheryl succinate, dl-α-tocopheryl acetate, combinations thereof, and the like; thiamin (vitamin B1), thiamin mononitrate, combinations thereof, and the like; riboflavin (vitamin B2), riboflavin 5′ monophosphate, combinations thereof, and the like; niacin (vitamin B3) from sources such as niacinamide, nicotinic acid, inositol hexanicotinate, combinations thereof, and the like; vitamin B6 from sources such as pyridoxine hydrochloride, pyridoxal-5′-phosphate, combinations thereof, and the like; folate from sources such as folic acid, folinic acid, combinations thereof, and the like; vitamin B12 from sources such as cyanocobalamin, methylcobalamin, combinations thereof, and the like; pantothenic acid (vitamin B5) from sources such as calcium d-pantothenate, pantetheine, pantothenol, sodium pantothenate, combinations thereof, and the like; biotin from sources such as biotin trituration and brewer's yeast, and the like, and combinations thereof.

Minerals that may be used in accordance with present disclosure include, for example: calcium from sources such as calcium carbonate, calcium citrate, calcium aspartate, calcium gluconate, calcium lactate, calcium lactate gluconate, calcium citrate malate, dicalcium malate, and the like, and combinations thereof chromium from sources such as chromium polynicotinate, chromium chloride, chromium-enriched yeast, chromium picolinate, chromium amino acid chelate, and the like, and combinations thereof copper from sources such as copper amino acid chelate, copper gluconate, copper sulfate, copper oxide, copper carbonate, and the like, and combinations thereof; iodine from sources such as potassium iodide, ammonium iodide, calcium iodide, kelp, and the like, and combinations thereof; iron from sources such as iron amino acid chelate, ferrous fumarate, ferrous succinate, ferrous sulfate, ferrous gluconate, ferritin, ferric orthophosphate, and the like, and combinations thereof; magnesium from sources such as magnesium oxide, magnesium carbonate, magnesium amino acid chelate; magnesium citrate, magnesium dolomite, magnesium aspartate, magnesium fumarate, magnesium hydroxide, dimagnesium malate, magnesium aspartate, and the like, combinations thereof; manganese from sources such as manganese amino acid chelate, manganese sulfate, manganese gluconate, manganese chloride, combinations thereof, and the like; molybdenum from sources such as sodium molybdate, molybdenum amino acid chelate, and the like, and combinations thereof; selenium from sources such as selenium yeast, selenomethionine, sodium selenite, combinations thereof, and the like; zinc from sources such as zinc oxide, zinc gluconate, zinc sulfate, zinc picolinate, zinc amino acid chelate, zinc chloride, and the like, and combinations thereof; boron from sources such as boron amino acid complex, sodium borate, and the like, and combinations thereof.

Herbals with various medicinal or dietary supplement properties may be used in the compositions in accordance with present disclosure. Herbals are generally aromatic plants or plant parts and/or extracts thereof that can be used medicinally or for flavoring. Suitable herbals can be used singly or in various mixtures. Commonly used herbs include echinacea, goldenseal, calendula, rosemary, thyme, kava, aloe, blood root, grapefruit seed extract, black cohosh, ginseng, guarana, cranberry, ginkgo biloba, St. John's wort, evening primrose oil, yohimbe bark, green tea, ma huang, maca, bilberry, lutein, and the like, and combinations thereof.

Any other dietary ingredient within the purview of those skilled in the art may be included with the confectionery nutritional supplements of the present disclosure. For example, in embodiments, coenzyme Q10 and/or omega III fatty acids may be included in the confectionery nutritional supplements of the present disclosure.

The vitamins and/or minerals described above may be provided and/or obtained in any form within the purview of those skilled in the art. The vitamins and/or minerals described above may also be obtained from any other source within the purview of those skilled in the art, including commercially available sources, and/or synthesized by methods within the purview of those skilled in the art.

It is also contemplated that drugs, medicaments, pharmaceuticals, antimicrobials, and the like, may be included in the compositions in accordance with the present disclosure. For example, tetrahydrocannabinol (THC) may be included in the compositions of the present disclosure at low dosage to provide analgesic effects. Various other drugs and medicaments, within the purview of those skilled in the art, which may be used in combination with the ingredients provided in the present disclosure include those in U.S. Pat. No. 7,727,565, the entire disclosure of which is hereby incorporated by reference in its entirety.

Coloring agents may also be used in the core or coating compositions of the present disclosure in amounts effective to produce a desired color. The coating composition described herein may provide a pearlescent appearance to a surface of the confectionery core. The pigment material may create a glossy finish appearance or a matte finish.

The pearlescent pigments used in the coating or core compositions are based on the formation of inorganic layers, or coatings, on substrates such as mica through the process of calcination. Examples of inorganic materials that may be used to form the inorganic layers include metal oxides such as titanium dioxide, iron oxide and combinations thereof. The core may be coated with one or more layers of the metal oxide. In one embodiment, the pearlescent pigment used in the coating composition includes at least one layer of titanium dioxide deposited on mica platelets.

In some embodiments, the particle size of the pearlescent pigment can be selected depending upon the desired visual effect to be attained. In embodiments, pearlescent pigments with an average particle size of less than 15 micrometers (μm) in diameter can create a silky-looking appearance. In embodiments, pearlescent pigments with an average particle size from about 5 μm to about 25 μm can create a satin-looking appearance. In embodiments, pearlescent pigments with an average particle size from about 10 μm to about 60 μm can create a metallic-looking appearance. In a further embodiment, pearlescent pigments with an average particle from about 10 μm to about 250 μm can create a glitter-looking appearance. Average particle sizes can be measured by any suitable methods known in the art. Specifically, particle size can be determined by any method known in the art such as those based upon sieve analysis using a standardized series of screens.

Pearlescent pigments can include materials such as those described in U.S. Patent Publication No. 2007/0014321 and U.S. Patent Publication No. 2008/0014321. Pearlescent pigments are commercially available under a number of trade names including SensiPearl™, Candurin™, and Edible Glitter™ (edible glitter). Pearlescent pigments such as those under the trade name of Candurin™ may provide a wide range of color effects including, for example, silver fine, silver sheen, silver luster, silver sparkle, gold shimmer, red shimmer, blue shimmer, green shimmer, gold sheen, light gold, gold luster, brown amber, orange amber, red amber, red luster and/or red sparkle.

In embodiments, edible gold fragments or flakes may be included in the core or coating compositions of the present disclosure in amounts effective to produce a desired color. In embodiments, the gold may be powdered or granulated. In embodiments, the gold may be edible 23-carat gold. In embodiments, the gold coatings are applied to the centers or core composition in any manner within the purview of the art, such as but not limited to spray coating.

The coloring agents in accordance with the present disclosure may further include only natural food coloring. Exemplary food coloring agents considered to be natural include, but are not limited to, annatto extract, (E160b), bixin, norbixin, astaxanthin, dehydrated beets (beet powder), beetroot red/betanin (E162), ultramarine blue, canthaxanthin (E161 g), cryptoxanthin (E161c), rubixanthin (E161d), violanxanthin (E161e), rhodoxanthin (E161f), caramel (E150(a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120); carmine (E132), indigo carmine, carmoisine/azorubine (E122), sodium copper chlorophyllin (E141), chlorophyll (E140), chlorophyllin, copper chlorophyllin, copper chlorophyll, curcumin/copper chlorophyllin, toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot, carrot oil, corn endosperm oil, paprika, paprika oleoresin, paprika extract, phaffia yeast, riboflavin (E101), saffron, turmeric (E100), turmeric oleoresin, tumeric root, amaranth (E123), capsanthin/capsorbin (E160c), lycopene (E160d), black carrot, black carrot extract, elderberry, aronia, allura red, curcumin, spinach, stinging nettle, burnt sugar, carbo medicinalis, and combinations thereof. In embodiments, the food coloring compositions may comprise a food coloring agent with at least one of titanium dioxide, sucrose, purified water, sodium carboxymethylcellulose, and combinations thereof.

In embodiments, methods of forming the compositions of the present disclosure include combining the at least one binding agent in at least one solvent to form a base solution into any suitable working chamber or silo. The base solution is warmed to a temperature from about 120 degrees Fahrenheit to about 300 degrees Fahrenheit to dissolve the binding agent in the solvent. At least one natural sweetener, at least one thickening agent, at least one stabilizing agent, at least one flavoring agent, and at least one natural active ingredient are then combined in a suitable mixer such as, a Hobart mixer with a paddle, for a time frame from about 5 minutes to about 120 minutes, or until the dry ingredients are well mixed to form a dry mixture. In embodiments, the base solution is then added to the dry mixture in a large mixer, for example one with a dough hook, and mixed for a time frame from about 5 minutes to about 120 minutes to form a doughy paste. Other suitable mixers and blenders are also contemplated for mixing the compositions of the present disclosure, such as a powder V mixer.

The doughy paste is then rolled with a dough roller to a desired thickness to form a sheet. In embodiments, the thickness of the doughy sheet may be from about 3 millimeters to about 50 millimeters, in embodiments from about 10 millimeters to about 40 millimeters.

The rolled dough is then placed into a die cutting machine to cut the sheet of dough into desired shapes. As mentioned above, various shapes are contemplated, such as pellets, tablets, capsules, balls, pillows, chunks, sticks slabs, stars, circles, squares, triangles, among others. Flatter and thinner shapes, such as the size of a quarter, may allow for quicker dissolving and absorption, while circular and thicker shapes, such as the size of a nickel, may provide for longer, lasting taste.

In embodiments, the mixed or rolled dough may be extruded through an extrusion die using any suitable methods known in the art, to obtain an extruded dough product in the desired shape. In embodiments, the dough may be pushed through an opening in the extrusion device that is about ½ inch thick at a desired pressure. The dough may be cut as it passes through the opening at about ¼ inches thickness and then placed onto a conveyer belt for the drying process.

The cut dough may then be placed into an oven to dry or be dried in a humid free environment at room temperature. In embodiments, the temperature of the oven may be from about 50 degrees Fahrenheit to about 400 degrees Fahrenheit, in embodiments at about 50 to 400 degrees Fahrenheit, for a time period from about 2 hours to about 72 hours. Other drying methods are also contemplated. The temperature and length of time may vary depending on the desired density, thickness of the die cut, or hardness of the dough. In embodiments, increasing the hardness or tensile strength of the dough may increase the delayed or extended release of an ingredient in the system. In embodiments, the dough may include about 2% to about 10% estimated moisture content.

The hardened dough or center is then subject to a coating process to add more flavor to the composition to form the confectionery supplements of the present disclosure. The coatings are intended to be hard or crunchy in nature. The coating may also allow for easy and safe storage of the supplements, longer shelf life of the supplements, and provide a more appealing look and taste. In embodiments, the coating process may include applying at least one sub coating layer and at least one sealing or wax layer to the core composition.

In embodiments, the core compositions or centers of the present disclosure may be coated with any natural coating syrups as an aqueous solution, including at least one natural sweetener in the form of a powder or syrup, a binding agent, any other additives if desired, such as, for example, a flavoring agent and/or coloring agent as provided herein, and combinations thereof. In embodiments, the coating layers may include any of the natural ingredients contemplated in accordance with the present disclosure. In embodiments, the binding agent may be gum arabic, the sweetener may be organic powdered sugar, and the coloring agent may be titanium dioxide.

In embodiments, the coating composition may be present in an amount from about 2% to about 60%, in embodiments, from about 20% to about 40% by weight of an individual center.

In embodiments, the natural sweetener may be present in the coating composition in an amount from about 30% to about 80% by weight of the coating, in embodiments from about 40% to about 60% by weight of the coating. In embodiments, the binding agent may be present in an amount from about 1% to about 15% by weight of the coating, in embodiments from about 5% to about 10% by weight of the coating. In embodiments, the coloring agent may be present in an amount from about 0.05% to about 10% by weight of the coating, in embodiments from about 1% to about 5% by weight of the coating with the remainder being water and minor amounts of optional additives, if employed.

Other components may be added in minor amounts to the coating and include flavoring agents, moisture adsorbing compounds, anti-adherent compounds, dispersing agents and film forming agents.

The flavoring agents may include any of the ingredients contemplated in the present disclosure. The moisture absorbing compounds suitable for use in the coating may include sugar alcohols such as xylitol or mannitol, dicalcium phosphate, and combinations thereof. Examples of useful anti-adherent compounds, which may also function as a filler, include talc, magnesium trisilicate and/or calcium carbonate. Examples of dispersing agents which may be employed in the coating include titanium dioxide, talc or any other anti-adherent compounds known in the art.

The coatings may be applied by pan coating, although other conventional techniques may be useful, such as for example, spray drying in a rotating drum. In pan coating methods, the centers are placed in a revolving coating pan and dusted using cool dry air. The coating syrup may be heated at temperatures from about 50 degrees Fahrenheit to about 400 degrees Fahrenheit and a portion thereof added to the revolving pan which has the centers therein. Generally, a single deposition of the coating syrup is not sufficient to provide the desired amount or thickness of coating deposited on the centers. In embodiments, it may be necessary to apply second, third or more coats of the coating syrup in order to build up the weight and thickness of the coating to desired levels. However, before applying subsequent layers of the coating syrup, the previously applied layers are allowed to dry by gently flowing warm air or by adding a drying agent such as calcium carbonate, or powdered sugar in the case of sugar coatings. In embodiments, the coating centers are removed from the pans for drying and the drying time between application of layers of coating(s) may be from about 2 hours to 72 hours. Thereafter, an additional coating of syrup is added followed by drying with air or drying agent and this procedure is repeated until the desired coating weight is obtained.

For example, in coating the centers, the applications of coating syrup are continued until the average confectionery weight reaches the required coating weight. Thus, if the coating is to have about 15% weight gain to about 25% weight gain due to the coating, application of 4 to 6 coats of coating syrup may be required. In embodiments, other types of syrups may be added between portions of the disclosed coating syrup such as a sugar solution. A flavoring agent such as peppermint oil, spearmint oil or the like may also be added. The distribution time, that is, the time during which the syrup or drying agent is mixed with the centers, may be from about 1 minute to about 3 minutes. After the desired weight of coating has been obtained, the coated centers are dried with cool air at a temperature from about 50 degrees Fahrenheit to about 80 degrees Fahrenheit.

In embodiments, the dried, coated centers may be processed through an automated weight machine which is configured to process values of the centers to determine the desired weight of each center and which sends any out-of-weight product to a specific container.

In embodiments, a sub coating layer including organic powdered sugar and gum arabic may be applied to the centers prior to application of any additional coating layers. In embodiments, the powdered sugar may be present in amounts of from about 1% to about 80% by weight of the composition, in embodiments from about 2% to about 60% by weight of the composition. In embodiments, the gum arabic may be present in amounts of from about 0.1% to about 10% by weight of the composition, in embodiments from about 1% to about 5% by weight of the composition.

In embodiments, the base coat may be applied in multiple layers to provide desired hardness and thickness. In embodiments, subsequent hard shell coating layers may be applied to the centers including sugar syrup in amounts from about 5% to about 40% by weight of the composition, in embodiments from about 10% to about 30% by weight of the composition, and/or a flavoring agent such as, for example, peppermint, may be applied in an amount from about 0.001% to about 15% by weight of the composition, in embodiments from about 0.1% to about 10% by weight of the composition. In embodiments, the hard shell coating may be applied in multiple layers to provide desired hardness and thickness.

In embodiments, a top wax coating layer including any edible wax component within the purview of those skilled in the art, may be applied to the coated centers to shine, smooth and/or seal the confectionery supplement. In embodiments, the wax coating includes carnuba wax and confectioners glaze in a liquid suspension. In embodiments, the carnuba wax is present in amounts from about 0.1% to about 5% by weight of the suspension, in embodiments from about 0.5% to about 4% by weight of the suspension, and the confectioners glaze is present in amounts from about 0.1% to about 5% by weight of the suspension, in embodiments from about 0.5% to about 4% by weight of the suspension. The confectionery supplements are allowed to dry and then may be packaged in small tins or plastic bags for easy transport.

It is comtemplated that the compositions of the present disclosure have a manual time release so that most or all of the active ingredients are absorbed by the body unlike most compositions or supplements on the market which only absorb about 40 to about 60% of the active ingredients. For example, when a 1000 mg of a typical supplement is taken, only about 400 mg to about 600 mg of the active ingredient is absorbed because of the binders and fillers which inhibit the absorption. Contrastly, in embodiments, the confectionary supplements of the present disclosure may be taken in an oral dosage form of about 1 to 2 confectionary supplements having about 100 to about 120 mg of active ingredient every hour for about 4 hours to about 10 hours, in embodiments, from about 5 hours to about 8 hours, as needed to alleviate symptoms and ailments described herein for about 80% to about 100% absorption, in embodiments from about 90% to about 99% absorption, for a total absorption of about 1000 mg to about 1200 mg of the active ingredients. The following Examples are being submitted to illustrate embodiments of the present disclosure. These Examples are intended to be illustrative only and are not intended to limit the scope of the present disclosure. Also, parts and percentages are by weight unless otherwise indicated.

### Example 1

A batch for making confectionery supplements of the present disclosure that provides increased energy and alertness was prepared with the ingredients listed below in Table 1.

TABLE 1

| | | % By Weight | Milligrams |
|---|---|---|---|
| | | of Total | (end |
| Ingredient | Grams | Composition | product) |
| Organic cane sugar powdered | 254.82 | 85.78 | 670.18 |
| Natural encapsulated caffeine | 10.15 | 3.416 | 26.69 |
| Powdered gum arabic | 17 | 5.722 | 10.36 |
| Water, tap | 0.587 | 1.97 | 44.71 |
| White rice syrup 26 DE | 9.994 | 3.364 | 26.28 |
| Gelatin 175 Bloom | 1.479 | .497 | 3.89 |
| Cocoa Extract, 3700 | 1.491 | 0.501 | 3.92 |
| Stevia, Reb A 97% | 0.17 | 0.05 | 0.45 |
| Peppermint Flavor | 1.36 | 0.457 | 3.58 |

The energy supplements were prepared by stirring the gelatin in water and heating the solution at a temperature of about 150 degrees Fahrenheit to dissolve the gelatin. The remaining dry ingredients in Table 1 above, i.e., cane sugar, caffeine, gum arabic, stevia, peppermint, cocoa, and the white rice syrup were combined in a Hobart mixer using a paddle. The gelatin solution was then added to the dry mixture and mixed in a large mixer with a dough hook to form a paste with dough-like consistency. The dough was then pushed through a dough roller to form a sheet of a desired thickness, i.e., about 3 mm to about 5 mm. The rolled dough was then placed into a die cutter to be cut into a desired shape and size which was suitable for easy consumption, i.e., in the form of a round tablet. The cut dough was placed on a tray and placed in an oven at a temperature of about 110 degrees Fahrenheit for about 48 hours to dry and harden providing centers with an estimated moisture content of about 2% to about 3%. In the present example, the weight of one tablet was about 1.1 grams (about 1100 mg). The caffeine content in the 1.1 gram confectionery tablet was about 33.80 milligrams.

### Example 2

The hardened centers formed in Example 1 were then subject to a 4 step coating process. The coatings were applied in layers to evenly coat the supplements, e.g. about 20 shots, utilizing a hard panning process as is typically used in the art of coating confectionaries.

A sub-coating was prepared with the ingredients listed below in Table 2. The confectionery supplements prior to the sub coating step were measured at 790 milligrams and 87.778% by weight of the total composition.

TABLE 2

| | | % By Weight of |
|---|---|---|
| Ingredient | Milligrams | Total Composition |
| Organic cane sugar powdered | 90 | 10 |
| Gum arabic solution, 25% | 20 | 2.222 |

The hardened centers were placed in a pan coating drum with a revolving pan rpm of 24. The sub-coating was applied to set and coat the confectionery supplements in the pan. Powdered sugar was applied to dry the centers. The centers were allowed to dry overnight on a metal pan tray in a humidity free room at room temperature.

The coating process was repeated with the ingredients listed below in Table 3. The centers prior to the second coating step were measured at 790 milligrams and 88.268% by weight of the composition.

TABLE 3

| | | % By Weight of |
|---|---|---|
| Ingredient | Milligrams | Total Composition |
| Organic cane sugar powdered | 90 | 10.056 |
| Gum arabic solution, 25% | 15 | 1.676 |

Powdered sugar was applied to dry the centers. The centers were allowed to dry overnight on a metal pan tray in a humidity free room at room temperature.

A hard shell coating was then applied with the ingredients listed below in Table 4. The centers prior to the hard shell coating step were measured at 948 milligrams and 75.574% by weight.

TABLE 4

| | | % By Weight of |
|---|---|---|
| Ingredient | Milligrams | Total Composition |
| Organic cane sugar syrup (70 brix) | 300 | 23.916 |
| Opacolor Nat cc150003 red carmine | 6 | 0.478 |
| Peppermint flavor, triple distilled | 0.4 | 0.032 |

The above 3 step coating process may provide an added weight of 21 grams of dried sugar shell to the centers. The centers were removed from the pan and allowed to dry overnight in a humidity free room at room temperature.

After drying overnight, the centers were placed back into a ribbed pan and about 0.37 grams of carnuba wax was applied thereon. The pan was tumbled for about 30 minutes to allow the wax to provide a glossy shine on the supplements. The supplements were then coated with about 0.40% by weight (about 1.5 grams) of confectioners glaze (available commercially from a number of vendors (e.g., Crystalac from Mantrose-Haeuser Co., Inc., Westport, Conn.; Temuss Products Canada Ltd., Ajax, Ontario, Canada), and can be prepared by following the manufacturer's instructions) to provide a very durable, hard, glossy finish. The confectionery supplements were allowed to dry overnight. The final weight of the dried confectionery supplements were about 1058 milligrams with a weight percent gain of about 14.93%. The confectionery supplements were then packaged.

### Example 3

A batch for making confectionery supplements of the present disclosure that provides weight loss benefits was prepared with the ingredients listed below in Table 5.

TABLE 5

| | | % By Weight | Milligrams |
|---|---|---|---|
| | | of Total | (end |
| Ingredient | Grams | Composition | product) |
| Organic cane sugar powdered | 213.979 | 69.265 | 484.88 |
| Garcinia Cambogia | 36.877 | 11.937 | 83.56 |
| Green Coffee Extract | 11.383 | 3.685 | 25.79 |
| Saffron | 3.938 | 1.275 | 6.19 |
| Powdered gum arabic | 17 | 5.66 | 8.92 |
| Water, tap | 25.527 | 8.263 | 57.84 |
| White rice syrup 26 DE | 9.994 | 3.235 | 22.65 |
| Gelatin 175 Bloom | 1.479 | 0.479 | 3.35 |
| Cocoa Extract, 3700 | 1.491 | 0.483 | 3.38 |
| Stevia, Reb A 97% | 0.17 | 0.055 | 0.39 |
| Peppermint Flavor | 1.36 | 0.440 | 3.08 |

The energy supplements were prepared by stirring the gelatin in water and heating the solution at a temperature of about 150 degrees Fahrenheit to dissolve the gelatin. The remaining dry ingredients in Table 5 above, i.e., cane sugar, garcinia cambogia, saffron, green coffee extract, gum arabic, stevia, peppermint, cocoa, and the white rice syrup were combined in a Hobart mixer with a paddle. The gelatin solution was then added to the dry mixture and mixed in a large mixer with a dough hook to form a paste with dough-like consistency. The dough was then pushed through a dough roller to form a sheet of a desired thickness, i.e., about 3 mm to about 5 mm. The rolled dough was then placed into a die cutter to be cut into a desired shape and size which is suitable for easy consumption, i.e., in the firm of a round tablet. The cut dough was placed on a tray and placed in an oven at a temperature of about 110 degrees Fahrenheit for about 12 hours to about 48 hours to dry and harden to form a center with an estimated moisture content of about 2% to about 3%. In the present example, the weight of one tablet was about 1.1 grams (about 1100 mg). The caffeine content in the 1.1 gram confectionery tablet was about 40.48 milligrams.

### Example 4

The hardened centers formed in Example 3 were then subject to a 4 step coating process. The coatings were applied in layers enough to evenly coat the centers, e.g. about 20 shots, utilizing a hard panning process as used in the art of coating confectionaries.

A sub-coating was prepared with the ingredients listed below in Table 6. The centers prior to the sub coating step were measured at 700 milligrams and 97.087% by weight of the total composition.

TABLE 6

| | | % By Weight of |
|---|---|---|
| Ingredient | Milligrams | Total Composition |
| Organic cane sugar powdered | 9 | 1.248 |
| Gum arabic solution, 25% | 12 | 1.664 |

The hardened centers were placed in a pan coating drum with a revolving pan rpm of 24. The sub-coating was applied to set and coat the centers in the pan. Powdered sugar was applied to dry the centers. The centers were allowed to dry overnight on a metal pan tray in a humidity free room at room temperature.

The coating process was repeated with the ingredients listed below in Table 7. The centers prior to the second coating step were measured at 700 milligrams and 87.391% by weight.

TABLE 7

| | | % By Weight of |
|---|---|---|
| Ingredient | Milligrams | Total Composition |
| Organic cane sugar powdered | 90 | 11.236 |
| Gum arabic solution, 25% | 11 | 1.373 |

Powdered sugar was applied to dry the centers. The centers were allowed to dry overnight on a metal pan tray in a humidity free room at room temperature.

A hard shell coating was then applied with the ingredients listed below in Table 8. The centers prior to the hard shell coating step were measured at 824 milligrams and 72.895% by weight.

TABLE 8

| | | % By Weight of |
|---|---|---|
| Ingredient | Milligrams | Total Composition |
| Organic cane sugar syrup (70 brix) | 300 | 26.539 |
| Titanium dioxide | 6 | 0.531 |
| Peppermint flavor, triple distilled | 0.4 | 0.035 |

The above 3 step coating process may provide an added weight of 21 grams of dried sugar shell to the centers. The centers were removed from the pan and allowed to dry overnight in a humidity free room at room temperature.

After drying overnight, the centers were placed back into a ribbed pan and about 0.37 grams of carnuba wax was applied thereon. The pan was tumbled for about 30 minutes to allow the wax to provide a glossy shine on the supplements. The supplements were then coated with about 0.40% by weight (about 1.5 grams) of confectioners glaze to provide a very durable, hard, glossy finish. The confectionery supplements were allowed to dry for about 12 hours to about 48 hours. The final weight of the dried confectionery supplements were about 904 milligrams with a weight percent gain of about 22.57%. The confectionery supplements were then packaged.

### Example 5

A batch for making confectionery supplements of the present disclosure that provides increased energy and alertness was prepared with the ingredients listed below in Table 9.

TABLE 9

| | Grams | % By Weight of |
|---|---|---|
| Ingredient | (quadruple x2) | Total Composition |
| Organic cane sugar powdered | 4825.30 | 85.783 |
| Natural encapsulated caffeine | 234.87 | 3.416 |
| (60% active) | | |
| Powdered gum arabic | 82.88 | 5.722 |
| Water, tap | 357.68 | .197 |
| White rice syrup 26 DE | 231.26 | 3.364 |
| Gelatin 175 Bloom | 31.12 | 0.497 |
| Cocoa Extract, 3700 | 31.36 | 0.501 |
| Stevia, Reb A 97% | 3.6 | 0.05722 |
| Peppermint Flavor | 28.64 | 0.457 |

The energy supplements were prepared by stirring the gelatin in water and heating the solution on a hot plat at a temperature of about 150 degrees Fahrenheit to about 175 degrees Fahrenheit to dissolve the gelatin. The remaining dry ingredients in Table 9 above, i.e., cane sugar, caffeine, gum arabic, stevia, and cocoa, were combined in a Hobart mixer using a paddle. The white rice syrup was added to the dry ingredients while mixing. The peppermint was added to the mixture slowly. The gelatin solution was then added to the dry mixture and mixed in a large mixer with a dough hook to form a paste with dough-like consistency. Water may be added as necessary to adjust for a dough-like consistency. Once the desired consistency was formed, the dough was removed and weighed. The dough was then pushed through a dough roller to form a sheet of a desired thickness, i.e., about 3 mm to about 5 mm. Powdered sugar was used as a dusting agent during the sheeting process. The rolled dough was then placed onto a clicker press cutting board and a die cutter was placed on top of the dough. The cutting board, the dough, and die cutter was placed in a clicker press and a pressure of about 65 psi to about 75 psi was activated to cut the dough into a desired shape and size which was suitable for easy consumption, i.e., in the form of a round tablet. The dough was then removed from the clicker press and any loose centers were removed by flipping the die over a tray. Any leftover uncut dough was removed to be recycled. The above steps were repeated until all the dough was cut. The cut dough was placed on a bakers rack and placed in an oven at a temperature of about 110 degrees Fahrenheit for about 48 hours to dry and harden providing centers with an estimated moisture content of about 2% to about 3%. In the present example, the weight of one tablet was about 1.1 grams (about 1100 mg). The caffeine content in the 1.1 gram confectionery tablet was about 33.80 milligrams.

### Example 6

The hardened centers formed in Example 5 were then subject to a 4 step coating process. The coatings were applied in layers to evenly coat the supplements, e.g. about 20 shots, utilizing a hard panning process as is typically used in the art of coating confectionaries.

A sub-coating was prepared with the ingredients listed below in Table 10. The confectionery supplements prior to the sub coating step were measured at 5050 grams and 87.778% by weight of the total composition.

TABLE 10

| | | % By Weight of |
|---|---|---|
| Ingredient | Grams | Total Composition |
| Organic cane sugar powdered | 575.30 | 10 |
| Gum arabic solution, 25% (made by | 126.56 | 2.222 |
| mixing 25% acacia gum with 75% water | | |
| for 30 mins) | | |

The hardened centers were placed in a pan coating drum with a revolving pan rpm of 24. The sub-coating was applied to set and coat the confectionery supplements in the pan. Powdered sugar was applied to dry the centers. The centers were allowed to dry overnight on a metal pan tray in a humidity free room at room temperature.

The coating process was repeated with the ingredients listed below in Table 11. The centers prior to the second coating step were measured at 5751 grams and 88.268% by weight of the composition.

TABLE 11

| | | % By Weight of |
|---|---|---|
| Ingredient | Grams | Total Composition |
| Organic cane sugar powdered | 655.17 | 10.056 |
| Gum arabic solution, 25% | 108.80 | 1.676 |

Powdered sugar was applied to dry the centers. The centers were allowed to dry overnight on a metal pan tray in a humidity free room at room temperature.

A hard shell coating was then applied with the ingredients listed below in Table 12. The centers prior to the hard shell coating step were measured at 6515 grams and 75.574% by weight.

TABLE 12

| | | % By Weight of |
|---|---|---|
| Ingredient | Grams | Total Composition |
| Organic cane sugar syrup (70 brix) | 8621.15 | 23.916 |
| Opacolor Nat cc150003 red carmine | 41.38 | 0.478 |
| Peppermint flavor, triple distilled | 2.76 | 0.032 |

The above 3 step coating process may provide an added weight of 21 grams of dried sugar shell to the centers. The centers were removed from the pan and allowed to dry overnight in a humidity free room at room temperature.

After drying overnight, the centers were placed back into a ribbed pan and about 0.8621 grams (0.01%) of carnuba wax was applied thereon. The pan was tumbled for about 30 minutes to allow the wax to provide a glossy shine on the supplements. The supplements were then coated with about 0.40% by weight (about 34.48 grams) of confectioners glaze (available commercially from a number of vendors (e.g., Crystalac from Mantrose-Haeuser Co., Inc., Westport, Conn.; Temuss Products Canada Ltd., Ajax, Ontario, Canada), and can be prepared by following the manufacturer's instructions) to provide a very durable, hard, glossy finish. The confectionery supplements were allowed to dry overnight. The final weight of the dried confectionery supplements were about 8656 grams with a weight percent gain of about 14.93%. The confectionery supplements were then packaged.

### Example 7

### Cholesterol/Weight Loss Study/Trial

A study was conducted utilizing confectionary supplements with a potential cholesterol and appetite suppressant/weight loss effect and having the following formula (“cholesterol burner supplements”):

TABLE 13

| | | % By Weight of |
|---|---|---|
| Ingredient | Grams | Total Composition |
| Evaporated Cane Juice | 213.979 | 69.265 |
| Berberine HCL | 36.877 | 11.937 |
| Picrorhiza Kurroa Root | 15.321 | 15.321 |
| Extract | | |
| White Rice Syrup 26 DE | 9.994 | 3.235 |
| Powdered gum arabic | 17 | 5.66 |
| Gelatin 175 Bloom | 1.479 | 0.479 |
| Cocoa Extract, 3700 | 1.491 | 0.483 |
| Peppermint Oil | 1.36 | 0.440 |
| Stevia, Reb A 97% | 0.17 | 0.055 |
| Yellow food coloring | .75 | .25 |
| (sucrose, purified water, | | |
| riboflavin, carmine, titanium | | |
| dioxide) | | |
| Confectioners glaze | 3.0 | 1.0 |

Twenty eight subjects at risk for diabetes mellitus, hypertension, dyslipidemia and/or other cardio-metabolic diseases were selected for inclusion in the cholesterol burner supplements study. The subjects were men or women of any racial background, with an age ≧25 years, and could be on existing antihypertensive, antidiabetic, or lipid lowering therapy. The study was a placebo-controlled, randomized, double blind study. The demographics and metabolic parameters of the study population were as follows:

TABLE 14

| | |
|---|---|
| Women, % | 61.5 |
| Age, y | 50.0 + 12.5 |
| SBP, mm Hg | 146.6 + 15.3 |
| DBF, mm Hg | 91.1 + 9.7 |
| Waist circumference (cm) | 108.4 + 15.8 |
| Total cholesterol, mg/dL | 226.6 + 39.9 |
| LDL cholesterol, mg/dL | 154.5 + 35.5 |
| HDL cholesterol, mg/dL | 43.0 + 10.0 |
| Triglycerides, mg/dL | 198.3 + 40.5 |
| Glucose, mg/dL | 105.7 + 17.3 |

Subjects were given the cholesterol burner supplements or a corresponding placebo to be taken three times per day before meals. The treatment period was for 12 weeks. Dietary counseling was given to all patients.

The primary effect studied in the trial was overall appetite. The secondary effects studied related to parameters of body fat, metabolic profile such as lipids and glucose, serum biomarkers, and blood pressure.

As illustrated above in Table 15, the cholesterol burner supplements had an overall effect on appetite suppression. Participants participated in a questionnaire after 12 weeks of therapy regarding the effects on parameters of appetite suppression and the results are provided in Table 16.

TABLE 16

| | Placebo | Cholesterol Burner |
|---|---|---|
| | Yes | Yes |
| 1. Reduced hunger between meals | 42% | 96% |
| 2. Feeling more energetic | 57% | 96% |
| 3. Eating less at mealtime | 42% | 100% |

As illustrated below in Table 17, the cholesterol burner supplement had an overall effect on parameters of body fat.

TABLE 17

| | Placebo | | Cholesterol Burner | | |
|---|---|---|---|---|---|
| | Baseline | 12 weeks | Baseline | 12 weeks | |
| Waist | | | | | |
| Circum- | | | | | |
| ference (cm) | | | | | |
| Male | 117 ± 27 | 121 ± 24 | 120 ± 20 | 111 ± 21 | p = 0.082 |
| Female | 108 ± 24 | 103 ± 19 | 110 ± 17 | 105 ± 20 | p = 0.114 |
| Total body | | | | | |
| fat | | | | | |
| (percentage) | | | | | |
| Male | 39.1 ± 6.6 | 39.2 ± 7.0 | 38.1 ± 5.5 | 35.3 ± 5.5 | p = 0.077 |
| Female | 42.2 ± 7.3 | 41.8 ± 6.5 | 42.8 ± 7.0 | 40.0 ± 6.1 | p = 0.098 |

As illustrated below in Table 18, the cholesterol burner supplement had an overall effect on LDL cholesterol levels.

As illustrated below in Table 19, the cholesterol burner supplement had an overall effect on systolic and diastolic blood pressure levels.

As illustrated below in Table 20, the cholesterol burner supplement had an overall effect on serum biomarker levels.

TABLE 20

| | Placebo | | Cholesterol Burner | |
|---|---|---|---|---|
| | Baseline | 12 weeks | Baseline | 12 weeks |
| Markers of | | | | |
| inflammation | | | | |
| PAI-1 (mg/ml) | 257 ± 47 | 236 ± 39 | 271 ± 35 | 189 ± 29 * |
| hsCRP (mg/l) | 2.1 ± 0.3 | 2.1 ± 0.4 | 2.4 ± 0.4 | 1.8 ± 0.3 |
| Markers of diabetes | | | | |
| and insulin | | | | |
| resistance | | | | |
| Glutathione redox | 3.0 ± 0.5 | 3.2 ± 0.4 | 3.1 ± 0.5 | 2.6 ± 0.4 * |
| ratio | | | | |
| HOMA-IR (index) | 2.3 ± 0.4 | 2.0 ± 0.4 * | 2.3 ± 0.3 | 1.5 ± 0.3 * |
| Direct measure- | | | | |
| ment of vas- | | | | |
| cular function | | | | |
| Endothelial FMD | 4.4 ± 0.7 | 4.8 ± 0.4 | 4.3 ± 0.8 | 9.5 ± 0.7 * |

* value differs (p < 0.05) from baseline and placebo/12 weeks (time and group comparison)

A summary of findings showed no adverse effects on liver or renal function. The supplements were well tolerated by the participants and were found to be safe. The study showed that the cholesterol burner supplements' effects met the primary endpoint (suppression of appetite and parameters of weight and diet) and several secondary endpoints. A rigorous protocol was followed utilizing maximal dosage of pharmaceutical management as indicated. The study was conducted on high risk participants and the efficacy of the cholesterol burner supplements was significant.

The findings indicated that the cholesterol burner supplements are a safe and effective agent in impacting individuals who are at risk for diabetes mellitus, hypertension, dyslipidemia (all of these are serious indicators of cardio and renovascular diseases) and can be used as adjunctive management or primary management for individuals who are unable or unwilling to take pharmaceutical drugs.

It will be understood that various modifications may be made to the embodiments disclosed herein. Therefore the above description should not be construed as limiting, but merely as exemplifications of useful embodiments. Those skilled in the art will envision other modifications within the scope and spirit of the claims appended hereto.

What is claimed is:

1. A composition comprising:
at least one natural sweetener in an amount from about 60% to about 95% by weight of the composition;
at least one binding agent;
at least one thickening agent;
at least one stabilizing agent;
at least one flavoring agent;
at least one natural active ingredient; and
at least one solvent.

2. The composition of claim 1, wherein the at least one natural sweetener is selected from the group consisting of organic evaporated cane sugar, beet sugar, stevia, sucrose, dextrose, fructose, glucose, corn syrup, maltose, maltodextrin, lactose, maple syrup, molasses, honey, barley malt, rice syrup, honey, agave nectar, fruit juice sweeteners, and combinations thereof.

3. The composition of claim 1, wherein the at least one natural sweetener comprises organic evaporated cane sugar and is present in an amount from about 60% to about 95% by weight of the composition.

4. The composition of claim 1, wherein the at least one binding agent is selected from the group consisting of gelatin, carrageenan, seaweed, locust bean gum, pectin, agar, alginate, guar gum, xanthan gum, gum arabic, starch derivatives, isomalt, and combinations thereof.

5. The composition of claim 1, wherein the at least one binding agent comprises gelatin having a Bloom rating of from about 150 to about 175 Bloom and is present in an amount from about 0.1% to about 3% by weight of the composition.

6. The composition of claim 1, wherein the at least one thickening agent is selected from the group consisting of white rice syrup, rice flour, rice starch, sucrose syrup, brown rice syrup, tapioca syrup, corn syrup, agave syrup, and combinations thereof.

7. The composition of claim 1, wherein the at least one thickening agent comprises white rice syrup in an amount from about 1% to about 8% by weight of the composition.

8. The composition of claim 1, wherein the at least one stabilizing agent is selected from the group consisting of gum arabic, maltodextrin, gelatin, carrageenan, seaweed, locust bean gum, pectin, agar, alginate, guar gum, xanthan gum, shellac, shellac gum, ascorbic acid, benzoic acid, fumaric acid, starch derivatives, and combinations thereof.

9. The composition of claim 1, wherein the at least one stabilizing agent comprises gum arabic in an amount from about 0.1% to about 5% by weight of the composition.

10. The composition of claim 1, wherein the at least one flavoring agent is selected from the group consisting of cocoa extract, spearmint, cinnamon, peppermint, wintergreen, berry mint, clove, bay, thyme, cedar leaf, nutmeg, sage, almond, vanilla, chocolate, coffee, lemon, orange, grape, lime, grapefruit, apple, pear, peach, strawberry, raspberry, blueberry, melon, mango, watermelon, cantaloupe, cherry, plum, pineapple, apricot, and combinations thereof.

11. The composition of claim 1, wherein the at least one flavoring agent comprises cocoa extract or peppermint in an amount from about 0.1% to about 10% by weight of the composition.

12. The composition of claim 1, wherein the at least one natural active ingredient is present in an amount from about 0.5% to about 25% by weight of the composition.

13. The composition of claim 1, wherein the at least one natural active ingredient aids in weight loss and is selected from the group consisting of garcinia cambogia, coffea robusta, green coffee extract, saffron extract, raspberry ketones, caffeine, green tea extract, hoodia gordonii, milk thistle, green tea, calcium, konjac root, reishi mushroom, fish oil, gymnema sylvestre, guggul, brindleberries, chitosan, whey protein, beta glucan, kunjo, African mango, alpha-cyclodextrin, iodine, vitamin D, n-acetyy-tyrosine, soy phospholipids, and combinations thereof.

14. The composition of claim 1, wherein the at least one natural active ingredient aids in increasing energy and is selected from the group consisting of caffeine extract, guarana, ginseng, ginko, yerba mate, green tea extract, taurine, kola nut extract, rhodiola root extract, minerals, vitamins, niacin, amino acids, and combinations thereof.

15. The composition of claim 1, wherein the at least one natural active ingredient aids in memory and brain function and is selected from the group consisting of phosphatidylcholine, caffeine, ginko extract, rhodiola root extract, vitamin B6, folic acid, vitamin B12, green tea extract, vinpocetine, theanine, blueberry powder, phosphatidylserine, dimethylaminoethanol, pantothenic acid, and combinations thereof.

16. The composition of claim 1, wherein the at least one natural active ingredient aids in immunity and is selected from the group consisting of vitamin D, vitamin C, zinc, magnesium, garlic, bee pollen, flax seed, nutritional yeast, alfalfa leaf, kelp, milk thistle, turmeric, ginger, thyme, suma root, wheat, soy, milk, jaborandi extract, iodine, and combinations thereof.

17. The composition of claim 1, wherein the at least one natural active ingredient aids in repairing bone loss and is selected from the group consisting of calcium, black walnut tincture, vitamin D, and combinations thereof.

18. The composition of claim 1, wherein the at least one natural active ingredient comprises garcinia cambogia, green coffee bean extract, saffron extract, encapsulated caffeine, and combinations thereof.

19. The composition of claim 1, wherein the at least one natural active ingredient alleviates symptoms of erectile dysfunction, enhances muscle growth, and/or lowers cholesterol and is selected from the group consisting of Berberine Hydrochloride HCl [97%], Picrorhiza kurroa, red ginseng, plant extracts, and combinations thereof.

20. The composition of claim 1, wherein the solvent is selected from the group consisting of water, white rice syrup, rice flour, rice starch, sucrose syrup, brown rice syrup, tapioca syrup, corn syrup, agave syrup, stevia syrup, and combinations thereof, in an amount from about 1% to about 20% by weight of the composition.

21. The composition of claim 1, further comprising at least one coating layer including at least one of component selected from the group consisting of sweeteners, binding agents, flavoring agents, coloring agents, waxes, and combinations thereof.

22. An edible confectionery formed from the composition of claim 1.

23. The edible confectionary according to claim 22, wherein absorption of the active ingredient is from 80% to about 100%.

24. A composition for forming a confectionery supplement comprising:
at least one natural sweetener in an amount from about 60% to about 90% by weight of the composition;
at least one binding agent in an amount from about 0.1% to about 3% by weight of the composition;
at least one thickening agent in an amount from about 1% to about 8% by weight of the composition;
at least one stabilizing agent in an amount from about 0.1% to about 5% by weight of the composition;
at least one flavoring agent in an amount from about 0.1% to about 10% by weight of the composition;
at least one natural active ingredient in an amount from about 0.5% to about 25% by weight of the composition; and
at least one solvent in an amount from about 1% to about 20% by weight of the composition.

25. A process of manufacturing confectionery supplements comprising:
combining at least one binding agent in at least one solvent to form a base solution;
combining at least one natural sweetener, at least one thickening agent, at least one stabilizing agent, at least one flavoring agent, and at least one natural active ingredient to form a dry mixture;
combining the dry mixture with the base solution to form a doughy paste;
rolling the doughy paste to a desired thickness;
cutting the rolled doughy paste into a desired shape;
drying the shaped doughy paste until hardened to a desired hardness to form a hardened dough;
applying at least one sub coating layer to the hardened dough to form the confectionery supplement; and
applying at least one outer layer to the sub coating layer to form the confectionery supplement.
